(19) United States
(12) Patent Application Publication
(10) Pub. No.: US 2006/0064137 A1
(43) Pub. Date: Mar. 23, 2006

(54) Method and system to control respiration by means of simulated action potential signals

(76) Inventor: Robert T. Stone, Sunnyvale, CA (US)

Correspondence Address: Ralph C. Francis;Francis Law Group, 1942 Embarcadero, Oakland, CA, 94606, US

(21) Appl. No.: 11/264,937

(22) Filed: Nov. 1, 2005

Related U.S. Application Data
(63) Continuation-in-part of application No. 11/129,264, filed on May 13, 2005.
Application No. 11/129,264 is a continuation-in-part of application No. 10/847,738, filed on May 17, 2004, now Pat. No. 6,937,903.
(60) Provisional application No. 60/471,104, filed on May 16, 2003.

(51) Int. Cl. A61N 1/18

(52) U.S. Cl. 607/41

(57) ABSTRACT

A method to control respiration generally comprising generating and transmitting at least one simulated action potential signal to the body that is recognizable by the respiratory system as a modulation signal.

### CROSS-REFERENCE TO RELATED APPLICATIONS

This application is a continuation-in-part of U.S. application Ser. No. 11/129,264, filed May 13, 2005, which is a continuation-in-part of U.S. application Ser. No. 10/847,738, filed May 17, 2004, which claims the benefit of U.S. Provisional Application No. 60/471,104, filed May 16, 2003.

### FIELD OF THE PRESENT INVENTION

The present invention relates generally to medical methods and systems for monitoring and controlling respiration. More particularly, the invention relates to a method and system for controlling respiration by means of simulated action potential signals.

### BACKGROUND OF THE INVENTION

As is well known in the art, the brain modulates (or controls) respiration via electrical signals (i.e., action potentials or waveform signals), which are transmitted through the nervous system. The nervous system includes two components: the central nervous system, which comprises the brain and the spinal cord, and the peripheral nervous system, which generally comprises groups of nerve cells (i.e., neurons) and peripheral nerves that lie outside the brain and spinal cord. The two systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain.

As is known in the art, a typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The cell body typically includes two types of outgrowths (or processes); the dendrites and the axon. Most neurons have several dendrites; these branch out in treelike fashion and serve as the main apparatus for receiving signals from other nerve cells.

The axon is the main conducting unit of the neuron. The axon is capable of conveying electrical signals along distances that range from as short as 0.1 mm to as long as 2 m. Many axons split into several branches, thereby conveying information to different targets.

Near the end of the axon, the axon is divided into fine branches that make contact with other neurons. The point of contact is referred to as a synapse. The cell transmitting a signal is called the presynaptic cell and the cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

Many nerves and muscles are involved in efficient respiration or breathing. The most important muscle devoted to respiration is the diaphragm. The diaphragm is a sheet-shaped muscle, which separates the thoracic cavity from the abdominal cavity.

With normal tidal breathing the diaphragm moves about 1 cm. However, in forced breathing, the diaphragm can move up to 10 cm. The left and right phrenic nerves activate diaphragm movement.

Diaphragm contraction and relaxation accounts for approximately 75% volume change in the thorax during normal quiet breathing. Contraction of the diaphragm occurs during inspiration. Expiration occurs when the diaphragm relaxes and recoils to its resting position. All movements of the diaphragm and related muscles and structures are controlled by coded electrical signals traveling from the brain.

Details of the respiratory system and related muscle structures are set forth in Co-Pending application Ser. No. 10/847,738, which is expressly incorporated by reference herein in its entirety.

The main nerves that are involved in respiration are the ninth and tenth cranial nerves, the phrenic nerve, and the intercostal nerves. The glossopharyngeal nerve (cranial nerve IX) innervates the carotid body and senses CO₂ levels in the blood. The vagus nerve (cranial nerve X) provides sensory input from the larynx, pharynx, and thoracic viscera, including the bronchi. The phrenic nerve arises from spinal nerves C3, C4, and C5 and innervates the diaphragm. The intercostal nerves arise from spinal nerves T7-11 and innervate the intercostal muscles.

The various afferent sensory neuro-fibers provide information as to how the body should be breathing in response to events outside the body proper.

An important respiratory control is activated by the vagus nerve and its preganglionic nerve fibers, which synapse in ganglia. The ganglia are embedded in the bronchi that are also innervated with sympathetic and parasympathetic activity.

It is well documented that the sympathetic nerve division can have no effect on bronchi or it can dilate the lumen (bore) to allow more air to enter during respiration, which is helpful to asthma patients, while the parasympathetic process offers the opposite effect and can constrict the bronchi and increase secretions, which can be harmful to asthma patients.

The electrical signals transmitted along the axon to control respiration, referred to as action potentials, are rapid and transient “all-or-none” nerve impulses. Action potentials typically have an amplitude of approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

A “neurosignal” is a composite signal that includes many action potentials. The neurosignal also includes an instruction set for proper organ function. A respiratory neurosignal would thus include an instruction set for the diaphragm to perform an efficient ventilation, including information regarding frequency, initial muscle tension, degree (or depth) of muscle movement, etc.

Neurosignals or “neuro-electrical coded signals” are thus codes that contain complete sets of information for complete organ function. As set forth in Co-Pending application Ser. No. 11/125,480, filed May 9, 2005, once these neurosignals, which are embodied in the “simulated action potential signals” referred to herein have been isolated, recorded, standardized and transmitted to a subject (or patient), a generated nerve-specific instruction (i.e., signal(s)) can be employed to control respiration and, hence, treat a multitude of respiratory system disorders. The noted disorders include, but are not limited to, sleep apnea, asthma, excessive mucus production, acute bronchitis and emphysema.

As is known in the art, sleep apnea is generally defined as a temporary cessation of respiration during sleep. Obstructive sleep apnea is the recurrent occlusion of the upper airways of the respiratory system during sleep. Central sleep apnea occurs when the brain fails to send the appropriate signals to the breathing muscles to initiate respirations during sleep. Those afflicted with sleep apnea experience sleep fragmentation and complete or nearly complete cessation of respiration (or ventilation) during sleep with potentially severe degrees of oxyhemoglobin desaturation.

Studies of the mechanism of collapse of the airway suggest that during some stages of sleep, there is a general relaxation of the muscles that stabilize the upper airway segment. This general relaxation of the muscles is believed to be a factor contributing to sleep apnea.

Various apparatus, systems and methods have been developed, which include an apparatus for or step of recording action potentials or coded electrical neurosignals, to control respiration and treat respiratory disorders, such as sleep apnea. The signals are, however, typically subjected to extensive processing and are subsequently employed to regulate a “mechanical” device or system, such as a ventilator. Illustrative are the systems disclosed in U.S. Pat. Nos. 6,360,740 and 6,651,652.

In U.S. Pat. No. 6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 6,651,652, a system and method for treating sleep apnea is disclosed. The noted system includes respiration sensor that is adapted to capture neuro-electrical signals and extract the signal components related to respiration. The signals are similarly processed and employed to control a ventilator.

A major drawback associated with the systems and methods disclosed in the noted patents, as well as most known systems, is that the control signals that are generated and transmitted are “user determined” and “device determinative”. The noted “control signals” are thus not related to or representative of the signals that are generated in the body and, hence, would not be operative in the control or modulation of the respiratory system if transmitted thereto.

It would thus be desirable to provide a method and system for controlling respiration that includes means for generating and transmitting simulated action potential signals to the body that are operative in the control of the respiratory system.

It is therefore an object of the present invention to provide a method and system for controlling respiration that overcomes the drawbacks associated with prior art methods and systems for controlling respiration.

It is another object of the present invention to provide a method and system for controlling respiration that includes means for generating and transmitting simulated action potential signals to the body that are operative in the control of the respiratory system.

It is another object of the invention to provide a method and system for controlling respiration that includes means for generating and transmitting simulated waveform or respiratory signals that substantially correspond to coded waveform signals that are generated in the body and are operative in the control of respiratory system.

It is another object of the invention to provide a method and system for controlling respiration that includes means for recording waveform signals that are generated in the body and operative in the control of respiration.

It is another object of the invention to provide a method and system for controlling respiration that includes processing means adapted to generate a base-line respiratory signal that is representative of at least one coded waveform signal generated in the body from recorded waveform signals.

It is another object of the invention to provide a method and system for controlling respiration that includes processing means adapted to compare recorded respiratory waveform signals to baseline respiratory signals and generate a respiratory signal as a function of the recorded waveform signal.

It is another object of the invention to provide a method and system for controlling respiration that includes monitoring means for detecting respiration abnormalities.

It is another object of the invention to provide a method and system for controlling respiration that includes a sensor to detect whether a subject is experiencing an apneic event

It is another object of the invention to provide a method and system for controlling respiration that can be readily employed in the treatment of respiratory system disorders, including sleep apnea, asthma, excessive mucus production, acute bronchitis and emphysema.

### SUMMARY OF THE INVENTION

In accordance with the above objects and those that will be mentioned and will become apparent below, the method to control respiration generally comprises (i) generating at least a first simulated action potential signal that is recognizable by the respiration system as a modulation signal and (ii) transmitting the first simulated action potential signal to the body to control the respiratory system.

In one embodiment of the invention, the simulated action potential signal has a first region having a first positive voltage in the range of approximately 100-1500 mV for a first period of time in the range of approximately 100-400 μsec and a second region having a first negative voltage in the range of approximately −50 mV to −750 mV for a second period of time in the range of approximately 200-800 μsec.

In a preferred embodiment of the invention, the first positive voltage is approximately 800 mV, the first period of time is approximately 200 μsec, the first negative voltage is approximately −400 mV and the second period of time is approximately 400 μsec.

In one embodiment of the invention, the simulated action potential signal is transmitted to the subject's nervous system. In another embodiment, the simulated action potential signal is transmitted proximate to a target zone on the neck, head or thorax.

In accordance with a further embodiment of the invention, the method for controlling respiration in a subject generally comprises (i) generating at least a first simulated action potential signal that is recognizable by the respiratory system as a modulation signal, (ii) monitoring the respiration status of the subject and providing at least one respiratory system status signal in response to an abnormal function of the respiratory system, (iii) transmitting the first simulated action potential signal to the body in response to a respiratory status signal that is indicative of respiratory distress or a respiratory abnormality.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A and 1B are illustrations of waveform signals captured from the body that are operative in the control of the respiratory system;

FIG. 2 is a schematic illustration of one embodiment of a respiratory control system, according to the invention;

FIG. 3 is a schematic illustration of another embodiment of a respiratory control system, according to the invention;

FIG. 4 is a schematic illustration of yet another embodiment of a respiratory control system, according to the invention;

FIGS. 5A and 5B are illustrations of simulated waveform signals that have been generated by the process means of the invention;

FIG. 6 is a schematic illustration of an embodiment of a respiratory control system that can be employed in the treatment of sleep apnea, according to the invention; and

FIG. 7 is a schematic illustration of one embodiment of a simulated action potential signal that has been generated by the process means of the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a respiratory disorder” includes two or more such disorders and the like.

### Definitions

The term “nervous system”, as used herein, means and includes the central nervous system, including the spinal cord, medulla, pons, cerebellum, midbrain, diencephalon and cerebral hemisphere, and the peripheral nervous system, including the neurons and glia.

The terms “waveform” and “waveform signal”, as used herein, mean and include a composite electrical signal that is generated in the body and carried by neurons in the body, including neurocodes, neurosignals and components and segments thereof.

The term “simulated waveform signal”, as used herein, means an electrical signal or component thereof that substantially corresponds to a “waveform signal”.

The term “simulated action potential signal”, as used herein, means and includes a signal that exhibits positive voltage (or current) for a first period of time and negative voltage for a second period of time. The term “simulated action potential signal” thus includes square wave signals, modified square wave signals and frequency modulated signals.

The term “signal train”, as used herein, means a composite signal having a plurality of signals, such as the “simulated action potential” and “simulated waveform” signals defined above.

Unless stated otherwise, the simulated action potential signals of the invention are designed and adapted to be transmitted continuously or at set intervals to a subject.

The term “respiration”, as used herein, means the process of breathing

The term “respiratory system”, as used herein, means and includes, without limitation, the organs subserving the function of respiration, including the diaphragm, lungs, nose, throat, larynx, trachea and bronchi, and the nervous system associated therewith.

The term “target zone”, as used herein, means and includes, without limitation, a region of the body proximal to a portion of the nervous system whereon the application of electrical signals can induce the desired neural control without the direct application (or conduction) of the signals to a target nerve.

The terms “patient” and “subject”, as used herein, mean and include humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The term “sleep apnea”, as used herein, means and includes the temporary cessation of respiration or a reduction in the respiration rate.

The terms “respiratory system disorder”, “respiratory disorder” and “adverse respiratory event”, as used herein, mean and include any dysfunction of the respiratory system that impedes the normal respiration process. Such dysfunction can be caused by a multitude of known factors and events, including spinal cord injury and severance.

The present invention substantially reduces or eliminates the disadvantages and drawbacks associated with prior art methods and systems for controlling respiration. In one embodiment of the invention, the method for controlling respiration in a subject generally comprises generating at least one simulated action potential signal that is recognizable by the subject's respiratory system as a modulation signal and transmitting the simulated action potential signal to the subject's body. In a preferred embodiment of the invention, the simulated action potential signal is transmitted to the subject's nervous system.

As indicated, neuro-electrical signals related to respiration originate in the respiratory center of the medulla oblongata. These signals can be captured or collected from the respiratory center or along the nerves carrying the signals to the respiratory musculature. The phrenic nerve has, however, proved particularly suitable for capturing the noted signals.

Methods and systems for capturing coded signals from the phrenic nerve(s), and for storing, processing and transmitting neuro-electrical signals (or coded waveform signals) are set forth in Co-Pending application Ser. Nos. 10/000,005, filed Nov. 20, 2001, and application Ser. No. 11/125,480 filed May 9, 2005; which are incorporated by reference herein in their entirety.

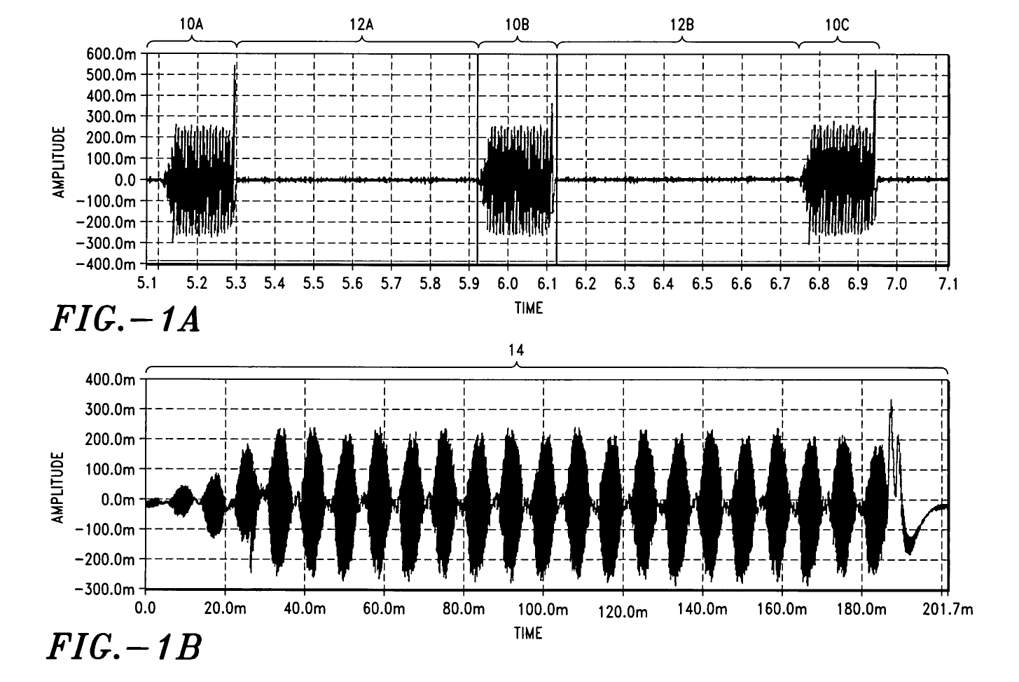

Referring first to FIGS. 1A and 1B, there are shown exemplar waveform signals that are operative in the efferent operation of the human (and animal) diaphragm; FIG. 1A showing three (3) signals 10A, 10B, 10C, having rest periods 12A, 12B therebetween, and FIG. 1B showing an expanded view of signal 10B. The noted signals traverse the phrenic nerve, which runs between the cervical spine and the diaphragm.

As will be appreciated by one having ordinary skill in the art, signals 10A, 10B, 10C will vary as a function of various factors, such as physical exertion, reaction to changes in the environment, etc. As will also be appreciated by one having skill in the art, the presence, shape and number of pulses of signal segment 14 can similarly vary from muscle (or muscle group) signal-to-signal.

As stated above, the noted signals include coded information related to inspiration, such as frequency, initial muscle tension, degree (or depth) of muscle movement, etc.

In accordance with one embodiment of the invention, neuro-electrical signals generated in the body that are operative in the control of respiration, such as the signals shown in FIGS. 1A and 1b, are captured and transmitted to a processor or control module.

Preferably, the control module includes storage means adapted to store the captured signals. In a preferred embodiment, the control module is further adapted to store the components of the captured signals (that are extracted by the processor) in the storage means according to the function performed by the signal components.

According to the invention, the stored signals can subsequently be employed to establish base-line respiration signals. The module can then be programmed to compare “abnormal” respiration signals (and components thereof) captured from a subject and, as discussed below, generate a simulated waveform signal or modified base-line signal for transmission to the subject. Such modification can include, for example, increasing the amplitude of a respiratory signal, increasing the rate of the signals, etc.

According to the invention, the captured neuro-electrical signals are processed by known means and a simulated waveform signal (i.e., simulated neuro-electrical coded signal) that is representative of at least one captured neuro-electrical signal and is operative in the control of respiration (i.e., recognized by the brain or respiratory system as a modulation signal) is generated by the control module. The noted simulated waveform signal is similarly stored in the storage means of the control module.

In one embodiment of the invention, to control respiration, the simulated waveform signal is accessed from the storage means and transmitted to the subject via a transmitter (or probe).

According to the invention, the applied voltage of the simulated waveform signal can be up to 20 volts to allow for voltage loss during the transmission of the signals. Preferably, current is maintained to less than 2 amp output.

Direct conduction into the nerves via electrodes connected directly to such nerves preferably have outputs less than 3 volts and current less than one tenth of an amp.

Referring now to FIG. 2, there is shown a schematic illustration of one embodiment of a respiratory control system 20A of the invention. As illustrated in FIG. 2, the control system 20A includes a control module 22, which is adapted to receive neuro-electrical coded signals or “waveform signals” from a signal sensor (shown in phantom and designated 21) that is in communication with a subject, and at least one treatment member 24.

The treatment member 24 is adapted to communicate with the body and receives the simulated waveform signal (or simulated action potential signal, discussed below) from the control module 22. According to the invention, the treatment member 24 can comprise an electrode, antenna, a seismic transducer, or any other suitable form of conduction attachment for transmitting respiratory signals that regulate or operate breathing function in human or animals.

The treatment member 24 can be attached to appropriate nerves or respiratory organ(s) via a surgical process. Such surgery can, for example, be accomplished with “key-hole” entrance in a thoracic-stereo-scope procedure. If necessary, a more expansive thoracotomy approach can be employed for more proper placement of the treatment member 24.

Further, if necessary, the treatment member 24 can be inserted into a body cavity, such as the nose or mouth, and can be positioned to pierce the mucinous or other membranes, whereby the member 24 is placed in close proximity to the medulla oblongata and/or pons. The simulated signals of the invention can then be sent into nerves that are in close proximity with the brain stem.

As illustrated in FIG. 2, the control module 22 and treatment member 24 can be entirely separate elements, which allow system 20A to be operated remotely. According to the invention, the control module 22 can be unique, i.e., tailored to a specific operation and/or subject, or can comprise a conventional device.

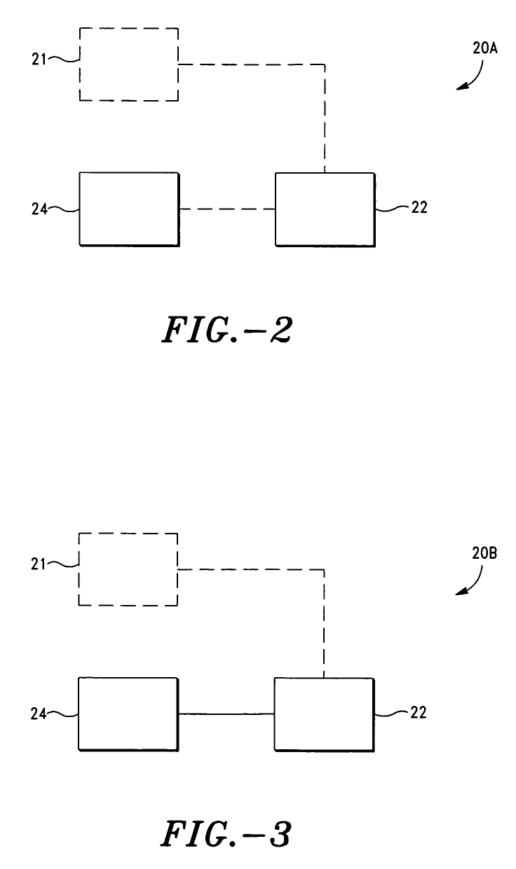

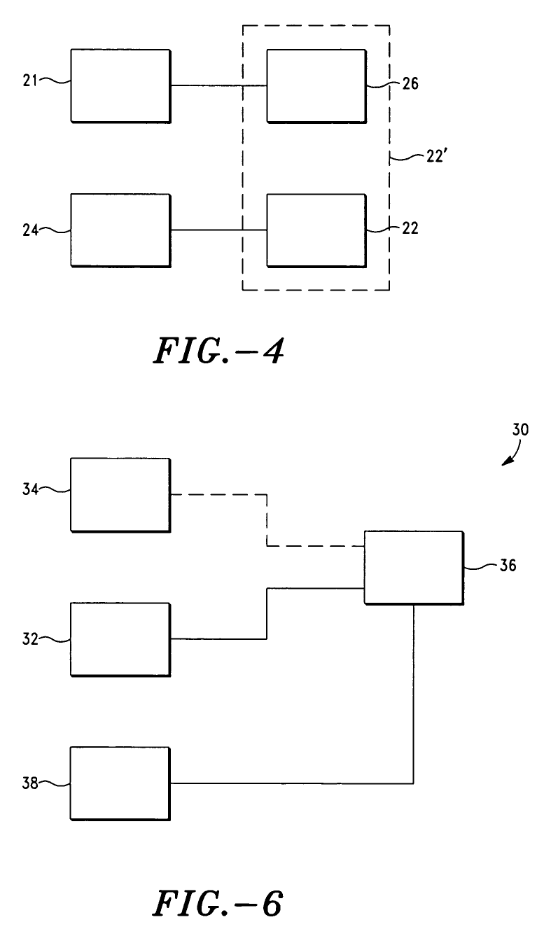

Referring now to FIG. 3, there is shown a further embodiment of a control system 20B of the invention. As illustrated in FIG. 3, the system 20B is similar to system 20A shown in FIG. 2. However, in this embodiment, the control module 22 and treatment member 24 are connected.

Referring now to FIG. 4, there is shown yet another embodiment of a control system 20C of the invention. As illustrated in FIG. 4, the control system 20C similarly includes a control module 22 and a treatment member 24. The system 20C further includes at least one signal sensor 21.

The system 20C also includes a processing module (or computer) 26. According to the invention, the processing module 26 can be a separate component or can be a sub-system of a control module 22′, as shown in phantom.

As indicated above, the processing module (or control module) preferably includes storage means adapted to store the captured respiratory signals. In a preferred embodiment, the processing module 26 is further adapted to extract and store the components of the captured respiratory signals in the storage means according to the function performed by the signal components.

According to the invention, in one embodiment of the invention, the method for controlling respiration in a subject includes generating a first simulated waveform signal that is recognizable by the respiratory system as a modulation signal and (ii) transmitting the first simulated waveform signal to the body to control the respiratory system.

In another embodiment of the invention, the method for controlling respiration comprises capturing coded waveform signals that are generated in a subject's body and are operative in the control of respiration, (ii) generating a first simulated waveform signal that is recognizable by the respiratory system as a modulation signal, and (iii) transmitting the first simulated waveform signal to the body.

In one embodiment of the invention, the first simulated waveform signal includes at least a second simulated waveform signal that substantially corresponds to at least one of the captured waveform signals and is operative in the control of the respiratory system.

In one embodiment of the invention, the first simulated waveform signal is transmitted to the subject's nervous system. In another embodiment, the first simulated waveform signal is transmitted proximate to a target zone on the neck, head or thorax.

According to the invention, the simulated waveform signals can be adjusted (or modulated), if necessary, prior to transmission to the subject.

In another embodiment of the invention, the method to control respiration generally comprises (i) capturing coded waveform signals that are generated in the body and are operative in control of respiration and (ii) storing the captured waveform signals in a storage medium, the storage medium being adapted to store the components of the captured waveform signals according to the function performed by the signal components, (iii) generating a first simulated waveform signal that substantially corresponds to at least one of the captured waveform signals, and (iv) transmitting the first simulated waveform signal to the body to the control the respiratory system.

In another embodiment of the invention, the method to control respiration generally comprises (i) capturing a first plurality of waveform signals generated in a first subject's body that are operative in the control of respiration, (ii) generating a base-line respiration waveform signal from the first plurality of waveform signals, (iii) capturing a second waveform signal generated in the first subject's body that is operative in the control of respiration, (iv) comparing the base-line waveform signal to the second waveform signal, (v) generating a third waveform signal based on the comparison of the base-line and second waveform signals, and (vi) transmitting the third waveform signal to the body, the third waveform signal being operative in the control of respiration.

In one embodiment of the invention, the first plurality of waveform signals is captured from a plurality of subjects.

In one embodiment of the invention, the step of transmitting the waveform signals to the subject's body is accomplished by direct conduction or transmission through unbroken skin at a selected appropriate zone on the neck, head, or thorax. Such zone will approximate a position close to the nerve or nerve plexus onto which the signal is to be imposed.

In an alternate embodiment of the invention, the step of transmitting the waveform signals to the subject's body is accomplished by direct conduction via attachment of an electrode to the receiving nerve or nerve plexus. This requires a surgical intervention to physically attach the electrode to the selected target nerve.

In yet another embodiment of the invention, the step of transmitting a signal to the subject's body is accomplished by transposing the signal into a seismic form. The seismic signal is then sent into a region of the head, neck, or thorax in a manner that allows the appropriate “nerve” to receive and obey the coded instructions of the seismic signal.

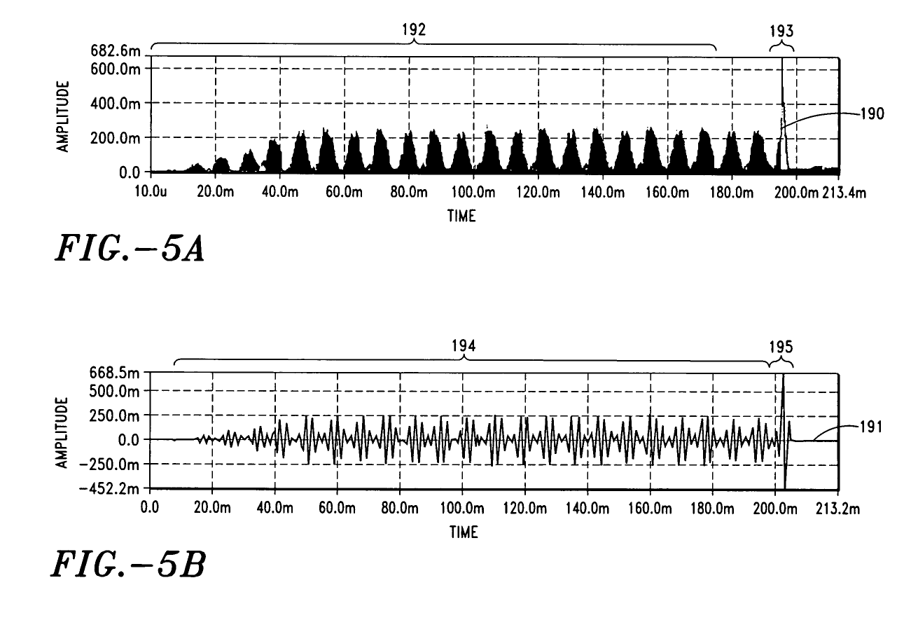

Referring now to FIGS. 5A and 5B, there are shown simulated waveform signals 190, 191 that were generated by the apparatus and methods of the invention. The noted signals are merely representative of the simulated waveform signals that can be generated by the apparatus and methods of the invention and should not be interpreted as limiting the scope of the invention in any way.

Referring first to FIG. 5A, there is shown the exemplar phrenic simulated waveform signal 190 showing only the positive half of the transmitted signal. The signal 190 comprises only two segments, the initial segment 192 and the spike segment 193.

Referring now to FIG. 5B, there is shown the exemplar phrenic simulated waveform signal 191 that has been fully modulated at 500 Hz. The signal 191 includes the same two segments, the initial segment 194 and the spike segment 195.

Referring now to FIG. 7, there is shown one embodiment of a simulated action potential signal 200 of the invention. As discussed in detail below, the simulated action potential signal 200 has been successfully employed to control respiration.

As illustrated in FIG. 7, the simulated action potential signal 200 comprises a modified, substantially square wave signal. According to the invention, the simulated action potential signal 200 includes a positive voltage region 202 having a first positive voltage (V₁) for a first period of time (T₁) and a first negative region 204 having a first negative voltage (V₂) for a second period of time (T₂).

Preferably, the first positive voltage (V₁) is in the range of approximately, 100-1500 mV, more preferably, in the range of approximately 700-900 mV, even more preferably, approximately 800 mV; the first period of time (T₁) is in the range of approximately 100-400 μsec, more preferably, in the range of approximately 150-300 μsec, even more preferably, approximately 200 μsec; the first negative voltage (V₂) is in the range of approximately, −50 mV to −750 mV, more preferably, in the range of approximately −350 mV to −450 mV, even more preferably, approximately −400 mV; the second period of time (T₂) is in the range of approximately 200-800 μsec, more preferably, in the range of approximately 300-600 μsec, even more preferably, approximately 400 μsec.

The simulated action potential signal 200 thus comprises a continuous sequence of positive and negative substantially square waves of voltage (or current) or bursts of positive and negative substantially square waves of voltage (or current), which preferably exhibits a DC component signal substantially equal to zero.

Preferably, the simulated action potential signal 200 has a repetition rate in the range of approximately 0.5-4 KHz, more preferably, in the range of approximately 1-2 KHz. Even more preferably, the repetition rate is approximately 1.6 KHz.

In a preferred embodiment of the invention, the maximum amplitude of the simulated action potential signal 200 is approximately 200 mV. As will be appreciated by one having ordinary skill in the art, the effective amplitude for the applied voltage is a strong function of several factors, including the electrode employed, the placement of the electrode and the preparation of the nerve.

According to the invention, the simulated action potential signals of the invention can be employed to construct “signal trains”, comprising a plurality of simulated action potential signals. The signal train can comprise a continuous train of simulated action potential signals or can included interposed signals or rest periods, i.e., zero voltage and current, between one or more simulated action potential signals.

The signal train can also comprise substantially similar simulated action potential signals, different simulated action potential signals or a combination thereof. According to the invention, the different simulated action potential signals can have different first positive voltage (V₁) and/or first period of time (T₁) and/or first negative voltage (V₂) and/or second period of time (T₂).

In accordance with one embodiment of the invention, the method for controlling respiration in a subject thus includes generating a first simulated action potential signal that is recognizable by the respiratory system as a modulation signal and (ii) transmitting the first simulated action potential signal to the body to control the respiratory system.

In one embodiment of the invention, the first simulated action potential signal is transmitted to the subject's nervous system. In another embodiment, the first simulated action potential signal is transmitted proximate to a target zone on the neck, head or thorax.

In accordance with a further embodiment of the invention, the method for controlling respiration in a subject includes generating a first signal train, said signal train including a plurality of simulated action potential signals that are recognizable by the respiratory system as modulation signals and (ii) transmitting the first signal train to the body to control the respiratory system.

According to the invention, the control of respiration can, in some instances, require sending simulated waveform and action potential signals into one or more nerves, including up to five nerves simultaneously, to control respiration rates and depth of inhalation. For example, the correction of asthma or other breathing impairment or disease involves the rhythmic operation of the diaphragm and/or the intercostal muscles to inspire and expire air for the extraction of oxygen and the dumping of waste gaseous compounds, such as carbon dioxide.

As is known in the art, opening (dilation) the bronchial tubular network allows for more air volume to be exchanged and processed for its oxygen content within the lungs. The dilation process can be controlled by transmission of the signals of the invention. The bronchi can also be closed down to restrict air volume passage into the lungs. A balance of controlling nerves for dilation and/or constriction can thus be accomplished through the methods and apparatus of the invention.

Further, mucus production, if excessive, can form mucoid plugs that restrict air volume flow throughout the bronchi. As is known in the art, no mucus is produced by the lung except in the lumen of the bronchi and also in the trachea.

The noted mucus production can, however, be increased or decreased by transmission of the signals of the invention. The transmission of the aforementioned signals of the invention can thus balance the quality and quantity of the mucus.

The present invention thus provides methods and apparatus to effectively control respiration rates and strength, along with bronchial tube dilation and mucinous action in the bronchi, by generating and transmitting simulated waveform and action potential signals to the body. Such ability to open bronchi will be useful for emergency room treatment of acute bronchitis or smoke inhalation injuries. Chronic airway obstructive disorders, such as emphysema, can also be addressed.

Acute fire or chemical inhalation injury treatment can also be enhanced through the methods and apparatus of the invention, while using mechanical respiration support. Injury-mediated mucus secretions also lead to obstruction of the airways and are refractory to urgent treatment, posing a life-threatening risk. Edema (swelling) inside the trachea or bronchial tubes tends to limit bore size and cause oxygen starvation. The ability to open bore size is essential or at least desirable during treatment.

Further, the effort of breathing in patients with pneumonia may be eased by modulated activation of the phrenic nerve through the methods and apparatus of the invention. Treatment of numerous other life threatening conditions also revolves around a well functioning respiratory system. Therefore, the invention provides the physician with a method to open bronchi and fine tune the breathing rate to improve oxygenation of patients. This electronic treatment method (in one embodiment) encompasses the transmission of activating or suppressing simulated action potential signals onto selected nerves to improve respiration. According to the invention, such treatments could be augmented by oxygen administration and the use of respiratory medications, which are presently available.

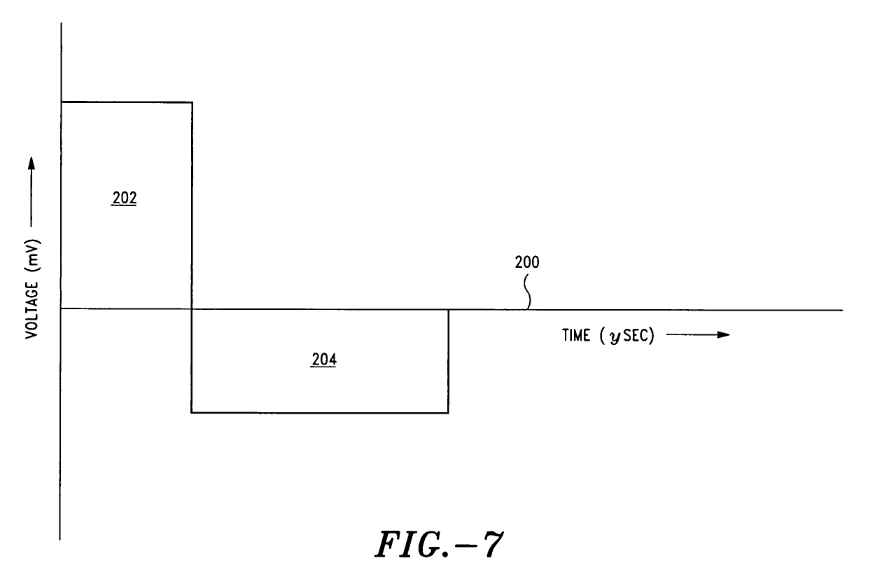

The methods and apparatus of the invention can also be effectively employed in the treatment of obstructive sleep apnea (or central sleep apnea) and other respiratory ailments. Referring now to FIG. 6, there is shown one embodiment of a respiratory control system 30 that can be employed in the treatment of sleep apnea. As illustrated in FIG. 6, the system 30 includes at least one respiration sensor 32 that is adapted to monitor the respiration status of a subject and transmit at least one signal indicative of the respiratory status.

According to the invention, the respiration status (and, hence, a sleep disorder) can be determined by a multitude of factors, including diaphragm movement, respiration rate, levels of O₂ and/or CO₂ in the blood, muscle tension in the neck, air passage (or lack thereof) in the air passages of the throat or lungs, i.e., ventilation. Various sensors can thus be employed within the scope of the invention to detect the noted factors and, hence, the onset of a respiratory disorder.

The system 30 further includes a processor 36, which is adapted to receive the respiratory system status signal(s) from the respiratory sensor 32. The processor 36 is further adapted to receive coded waveform signals recorded by a respiratory signal probe (shown in phantom and designated 34).

In a preferred embodiment of the invention, the processor 36 includes storage means for storing the captured, coded waveform signals and respiratory system status signals. The processor 36 is further adapted to extract the components of the waveform signals and store the signal components in the storage means.

In a preferred embodiment, the processor 36 is programmed to detect respiratory system status signals indicative of respiration abnormalities and/or waveform signal components indicative of respiratory system distress and generate at least one simulated waveform signal or simulated action potential signal that is operative in the control of respiration.

Referring to FIG. 6, the simulated waveform signal or simulated action potential signal is routed to a transmitter 38 that is adapted to be in communication with the subject's body. The transmitter 38 is adapted to transmit the simulated waveform signal or simulated action potential signal to the subject's body (in a similar manner as described above) to control and, preferably, remedy the detected respiration abnormality.

According to the invention, the simulated waveform signal or simulated action potential signal is preferably transmitted to the phrenic nerve to contract the diaphragm, to the hypoglossal nerve to tighten the throat muscles and/or to the vagus nerve to maintain normal brainwave patterns. A single signal or a plurality of signals can be transmitted in conjunction with one another.

Thus, in accordance with a further embodiment of the invention, the method for controlling respiration in a subject generally comprises (i) generating at least a first simulated action potential signal that is recognizable by the respiratory system as a modulation signal, (ii) monitoring the respiration status of the subject and providing at least one respiratory system status signal in response to an abnormal function of the respiratory system, (iii) transmitting the simulated action potential signal to the body to control the respiration system in response to a respiration status signal that is indicative of respiratory distress or a respiratory abnormality.

### EXAMPLES

The following examples are provided to enable those skilled in the art to more clearly understand and practice the present invention. They should not be considered as limiting the scope of the invention, but merely as being illustrated as representative thereof.

### Example 1

Four (4) juvenile swine, ranging in weight from 40 to 80 lbs., were exposed to nebulized methacholine that was dissolved in saline. Ventilation parameters, arterial oxygen saturation and exhaled carbon dioxide were monitored at various concentrations of methacholine.

The vagus nerve of the swine was exposed in the neck. As reflected in Table I, three signals were employed. Signal 1 comprised a sinusoidal signal having 500 Hz at 800 mV. Signal 2 comprised a simulated action potential signal having a 400 μsec, 800 mV positive voltage region and a 800 μsec, −400 mV negative voltage region. Signal 3 comprised a simulated action potential signal having a 200 μsec, 800 mV positive voltage region and a 400 μsec, −400 mV negative voltage region.

TABLE I

| Parameter | Methacholine | Signal 1 | Signal 2 | Signal 3 |
|---|---|---|---|---|
| Tidal | Increased | No Effect | No Effect | Decreased |
| Volume | | | | |
| Respiration | Decreased | Decreased | Decreased | Greatly |
| Rate | | | | Decreased |
| Inspiratory | Increased | Decreased | Decreased | Greatly |
| Pressure | | | | Decreased |
| Manual | Yes, 20 | Yes, | Yes, | No, no |
| Ventilation | seconds to | increased | increased | adverse effect |
| required | recover | recovery | recovery | observed |
| | | time | time | |

Referring to Table I, it can be seen that, upon administration of methacholine and transmittal of the noted signals, there was a marked reduction in respiratory rate and effort, which were similar to baseline levels without administration of methacholine. There was also a marked reduction in oxygen saturation and exhaled CO₂.

It was further found that when a simulated action potential signal having a first positive voltage of 800 mV for 200 μsec and a first negative voltage of approximately −400 mV for approximately 400 μsec was applied to the swine, a reduction in sensitivity to methacholine of at least a factor of 2, and as much as a factor of 8, was realized.

The example thus reflects that a modified square wave signal can be applied to the vagus nerve to dramatically reduce the physiologic response to drugs that produce asthma symptoms. As will be appreciated by one having ordinary skill in the art, the simulated action potential signals of the invention can thus be effectively employed to mitigate the normal human response to asthma triggers, reduce the severity of asthma attacks and permit delivery of anti-inflammatory medication for better control of asthma symptoms during acute attacks.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for controlling respiration in a subject, comprising the steps of:
generating a first simulated action potential signal that is recognizable by the subject's respiratory system as a modulation signal; and
transmitting at least the first simulated action potential signal to the subject's body, whereby control of the subject's respiratory system is effectuated.

2. The method of claim 1, wherein said first simulated action potential signal includes a positive voltage region having a first positive voltage for a first period of time and a first negative region having a first negative voltage for a second period of time.

3. The method of claim 2, wherein said first positive voltage is in the range of approximately 100-1500 mV.

4. The method of claim 2, wherein said first positive voltage is in the range of approximately 700-900 mV.

5. The method of claim 2, wherein said first positive voltage is approximately 800 mV.

6. The method of claim 2, wherein said first period of time is in the range of approximately 100-400 μsec.

7. The method of claim 2, wherein said first period of time is in the range of approximately 150-300 μsec.

8. The method of claim 2, wherein said first period of time is approximately 200 μsec.

9. The method of claim 2, wherein said first negative voltage is in the range of approximately −50 mV to −750 mV.

10. The method of claim 2, wherein said first negative voltage is in the range of approximately −350 mV to −450 mV.

11. The method of claim 2, wherein said first negative voltage is approximately −400 mV.

12. The method of claim 2, wherein said second period of time is in the range of approximately 200-800 μsec.

13. The method of claim 2, wherein said second period of time is in the range of approximately 300-600 μsec.

14. The method of claim 2, wherein said second period of time is approximately 400 μsec.

16. The method of claim 1, wherein said simulated action potential signal is transmitted to the subject's nervous system.

17. The method of claim 1, wherein the subject comprises a human.

18. The method of claim 1, wherein the subject comprises an animal.

19. A method for controlling respiration, comprising the steps of:
monitoring the respiration status of a subject and providing at least one respiratory system status signal indicative of the status of the subject's respiratory system;
generating a first simulated action potential signal that is recognizable by the subject's respiratory system as a modulation signal; and
transmitting said first square wave signal to said subject in response to said respiratory system status signal.

20. The method of claim 19, wherein said first simulated action potential signal includes a positive voltage region having a first positive voltage for a first period of time and a first negative region having a first negative voltage for a second period of time.

21. The method of claim 20, wherein said first positive voltage is in the range of approximately 100-1500 mV.

22. The method of claim 20, wherein said first positive voltage is in the range of approximately 700-900 mV.

23. The method of claim 20, wherein said first positive voltage is approximately 800 mV.

24. The method of claim 20, wherein said first period of time is in the range of approximately 100-400 μsec.

25. The method of claim 20, wherein said first period of time is in the range of approximately 150-300 μsec.

26. The method of claim 20, wherein said first period of time is approximately 200 μsec.

27. The method of claim 20, wherein said first voltage is in the range of approximately −50 mV to −750 mV.

28. The method of claim 20, wherein said first negative voltage is in the range of approximately −350 mV to −450 mV.

29. The method of claim 20, wherein said first negative voltage is approximately −400 mV.

30. The method of claim 20, wherein said second period of time is in the range of approximately 200-800 μsec.

31. The method of claim 20, wherein said second period of time is in the range of approximately 300-600 μsec.

32. The method of claim 20, wherein said second period of time is approximately 400 μsec.

33. The method of claim 19, wherein said first simulated action potential signal is transmitted to said subject's nervous system.

34. The method of claim 19, wherein said first simulated action potential signal is transmitted to a target zone on said subject, said target zone being selected from the neck, head and thorax.

35. The method of claim 19, wherein said subject comprises a human.

36. The method of claim 19, wherein said subject comprises an animal.
